(19) United States
(12) Patent Application Publication
(10) Pub. No.: US 2006/0178684 A1
(43) Pub. Date: Aug. 10, 2006

(54) Method and apparatus for supporting a surgical component

(76) Inventors: Hugh H. Trout III, Bethesda, MD (US); Howard M. Tanner, Naperville, IL (US)

Correspondence Address: KELLEY DRYE & WARREN LLP, 3050 K STREET, NW, SUITE 400, WASHINGTON, DC, 20007, US

(21) Appl. No.: 11/326,351

(22) Filed: Jan. 6, 2006

Related U.S. Application Data
(63) Continuation of application No. 10/424,775, filed on Apr. 29, 2003.
(60) Provisional application No. 60/375,805, filed on Apr. 29, 2002.

(51) Int. Cl. A61B 17/08

(52) U.S. Cl. 606/153

(57) ABSTRACT

The present invention relates generally to a surgical support apparatus and method for use in a surgical procedure. In particular, the present invention relates to devices and methods that provide support to a surgical component during attachment of the component during a surgical procedure. The various embodiments of the present invention include components that apply force to the surgical component bringing the component into contact with another surgical component or a tissue, such as, but not limited to, an intimal vessel wall. These components may include an expandable cuff, and an expandable cuff with at least one attachment element, fin elements, a ring assembly with at least two helical strands, or with at least one spoke.

### CROSS-REFERENCE TO RELATED APPLICATIONS

The present invention is a continuation of U.S. patent application Ser. No. 10/424,775, filed on Apr. 29, 2003 which relates to, and is entitled to the benefit of the earlier filing date and priority of U.S. Provisional Application Ser. No. 60/375,805, filed on Apr. 29, 2002.

### FIELD OF THE INVENTION

The present invention relates generally to a surgical support apparatus and method for use in a surgical procedure. In particular, the present invention relates to devices and methods that provide temporary support to a surgical component for attachment of the component to tissue or to another surgical component during a surgical procedure.

### BACKGROUND OF THE INVENTION

An aneurysm is a ballooning of the wall of an artery resulting from the weakening of the artery due to disease or other conditions. Left untreated, the aneurysm will frequently rupture, resulting in loss of blood through the rupture and death.

Aortic aneurysms are the most common form of arterial aneurysm and are life threatening. The aorta is the main artery which supplies blood to the circulatory system. The aorta arises from the left ventricle of the heart, passes upward and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

It is common for an aortic aneurysm to occur in that portion of the abdominal aorta between the renal arteries and the iliac arteries. This portion of the abdominal aorta is particularly susceptible to weakening, resulting in an aortic aneurysm. Such an aneurysm is often located near the iliac arteries. An aortic aneurysm larger than about 5 cm in diameter in this section of the aorta is ominous. Left untreated, the aneurysm may rupture, resulting in rapid, and usually fatal, hemorrhaging. Typically, a surgical procedure is not performed on aneurysms smaller than 5 cm because no statistical benefit exists in performing such procedures.

Aneurysms in the abdominal aorta are associated with a particularly high mortality rate; accordingly, current medical standards call for urgent operative repair. Abdominal surgery, however, results in substantial stress to the body. Although the mortality rate for an aortic aneurysm is high, there is also considerable mortality and morbidity associated with open surgical intervention to repair an aortic aneurysm. This intervention involves penetrating the abdominal wall to the location of the aneurysm to reinforce or replace the diseased section of the aortic aneurysm. A prosthetic device, typically a synthetic tube graft, is used for this purpose. The graft serves to exclude the aneurysm from the circulatory system, thus relieving pressure and stress on the weakened section of the aorta at the aneurysm.

Repair of an aortic aneurysm by surgical means is a major operative procedure. Substantial morbidity accompanies the procedure, resulting in a protracted recovery period. Further, the procedure entails a substantial risk of mortality. While surgical intervention may be indicated and the surgery carries attendant risk, certain patients may not be able to tolerate the stress of intra-abdominal surgery. It is, therefore, desirable to reduce the mortality and morbidity associated with intra-abdominal surgical intervention.

In recent years, methods have been developed to attempt to treat an aortic aneurysm without the attendant risks of intra-abdominal surgical intervention. Among them are inventions disclosed and claimed in Kornberg, U.S. Pat. No. 4,562,596 for Aortic Graft, Device and Method for Performing an Intraluminal Abdominal Aortic Aneurysm Repair; Lazarus, U.S. Pat. No. 4,787,899 for Intraluminal Graft Device, System and Method; and Taheri, U.S. Pat. No. 5,042,707 for Intravascular Stapler, and Method of Operating Same.

Although in recent years certain techniques have been developed that may reduce the stress, morbidity, and risk of mortality associated with surgical intervention to repair aortic aneurysms, including delivery catheter assemblies, none of the systems that have been developed provide an apparatus and method that supports a surgical component during a surgical procedure in the manner of the embodiments of the present invention. An embodiment of the present invention provides an apparatus and method for supporting a surgical component, such as a prosthetic graft, during attachment of the component at a surgical site, particularly to a vessel wall or to another component. In particular, the apparatus according to an embodiment of the present invention forces the surgical component into contact with the vessel wall and maintains the support during attachment of the component to the vessel wall. Further, the apparatus of an embodiment of the present invention can be controlled by an interventionist outside the body, which reduces the intrusiveness of the surgical procedure.

It is therefore an advantage of some, but not necessarily all, embodiments of the present invention to provide an apparatus and method for facilitating the repair of aortic aneurysms. It is another advantage of an embodiment of the present invention to provide an apparatus and method for supporting a surgical component in position for attachment during a surgical procedure. It is yet another advantage of an embodiment of the present invention to provide an apparatus and method for supporting a surgical component that may be controlled outside the body by an interventionist.

Additional advantages of various embodiments of the invention are set forth, in part, in the description that follows and, in part, will be apparent to one of ordinary skill in the art from the description and/or from the practice of the invention.

### SUMMARY OF THE INVENTION

Responsive to the foregoing challenges, Applicant has developed an innovative apparatus for supporting a surgical component during a surgical procedure. An embodiment of the present invention is an apparatus for supporting a surgical component, comprising an expandable cuff and expansion means for expanding the cuff to contact a surface to support a surgical component. The surface may comprise tissue, and/or the surgical component, and/or another surgical component. The expandable cuff may be incorporated into the surgical component, or may be attached to the surgical component by at least one attachment element. The expandable cuff may be removably attached to the surgical component. The expandable cuff may be inflatable and may have a lumen attached to the expandable cuff. In alternative embodiments, the expandable cuff may comprise a ring assembly, further comprising at least two ring elements and at least two helical strands, and/or a tubular element and fins, and/or spokes and a conical tip.

In an alternative embodiment, the apparatus for supporting a surgical component during a surgical procedure, may comprise an expandable cuff, wherein the expandable cuff is inflatable, a lumen with a first end and a second end, wherein the first end is attached to the expandable cuff, and expansion means attached to the second end of the lumen for expanding the expandable cuff into contact with a surface to support a surgical component. The expandable cuff may be incorporated into the surgical component or attached to the surgical component by at least one attachment element.

In another embodiment, the apparatus for supporting a surgical component during a surgical procedure may comprise an expandable cuff comprising a ring assembly and expansion means for expanding the cuff to contact a surface to support a surgical component. The ring assembly may further comprise a first and a second ring element and at least two helical strands with a first end and a second end, wherein the first end is attached to the first ring element and the second end is attached to the second ring element.

In an alternative embodiment, the apparatus for supporting a surgical component during a surgical procedure may comprise an expandable cuff comprising a tubular element and at least one fin, wherein the at least one fin has a first end and a second end, wherein the first end is substantially free and passes through an opening in the tubular element and expansion means attached to the second end of the at least one fin for expanding the first end of the at least one fin into contact with a surface to support a surgical component.

In an alternative embodiment, the apparatus for supporting a surgical component during a surgical procedure may comprise an expandable cuff comprising a conical tip and a spoke assembly with a first and a second configuration, wherein the spoke assembly is disposed within the conical tip in the first configuration, wherein the spoke assembly further comprises at least one spoke with a first end and a second end, wherein the first end is substantially free, and expansion means for expanding the spoke assembly into a second configuration wherein the first end of the at least one spoke contacts a surface to support a surgical component. The second end of the at least one spoke may be attached to the expansion means, and/or the conical tip may be attached to the expansion means.

An embodiment of the present invention is a method for supporting a surgical component for placement at a surgical site, comprising the steps of gaining access to a surgical site, delivering a support apparatus to the surgical site, delivering a surgical component to the surgical site, expanding an expandable cuff of the support apparatus into contact with a surface, and supporting the surgical component at the surgical site during a surgical procedure. The support apparatus and surgical component may be attached and delivered to the surgical site in the same step. The step of expanding the expandable cuff may comprise the step of inflating the expandable cuff, drawing at least two ring elements together to expand at least two helical strands into contact with a surface, expanding at least one fin into contact with a surface. An alternative embodiment of the method may further comprise the step of contracting the expandable cuff to its pre-expansion configuration. An alternative embodiment of the method may further comprise the step of removing the support apparatus from the surgical site following the surgical procedure. An alternative embodiment of the method may further comprise the step of attaching the surgical component to a tissue, and/or to another surgical component.

Another embodiment of the apparatus for supporting a surgical component comprises a surgical component having a distal neck, an expandable cuff located at the distal neck of the surgical component, and an integrated lumen within the surgical component leading to the cuff for inflating the cuff. The cuff may be inflated by a compressed gas, compressed gas mixture, or a fluid.

Another embodiment of the apparatus for supporting a surgical component comprises a surgical component, an expandable cuff held in direct but loose association with the surgical component by at least one attachment element element, or in an alternative embodiment, a plurality of finger elements, that may be rigid in nature, and a lumen extending between a point of distal egress and the cuff for inflating the cuff. The apparatus may further comprise detailing at a central axis of the cuff for supporting related surgical members during a surgical procedure. Alternatively the apparatus may comprise detailing at a central axis of the cuff having related surgical components attached thereto during a surgical procedure.

Another alternative embodiment of the apparatus for supporting a surgical component comprises a tubular element and a plurality of fin elements attached about an external surface of the tubular element.

Another embodiment of the apparatus for supporting a surgical component comprises a tubular element and a ring assembly adjustably attached to the tubular element, the assembly comprising two or more ring elements connected by a plurality of helical strands. The ring assembly comprises a biocompatible material. The ring assembly may comprise plastic or metal material, or any other suitable material.

Yet another embodiment of the apparatus for supporting a surgical component comprises a tubular installation element having a stepped diameter, a positioning cuff slidably located on the tubular element, the cuff having a plurality of spokes that radiate from the tubular element, and a substantially hollow conical tip for encapsulating the cuff. The positioning cuff may comprise a plastic or metal material or any other suitable biocompatible material. Further, the positioning cuff may comprise a single component or an assembly of components.

An embodiment of the present invention is also directed to a method for supporting a surgical component for placement at a surgical site, comprising the steps of gaining access to a surgical site, delivering a surgical component having an expandable cuff at a distal neck of the component to the surgical site, inflating the cuff through an integrated lumen within the surgical component, thereby expanding the surgical component into contact with an intimal wall of a vessel, attaching the surgical component to the vessel wall, and thereafter deflating the cuff.

Another method for supporting a surgical component for placement at a surgical site, comprises the steps of gaining access to a surgical site, delivering a surgical component having an expandable cuff at a distal neck of the component to the surgical site, inflating the cuff through an integrated lumen within the surgical component, thereby expanding the surgical component into contact with an intimal wall of a vessel, attaching the surgical component to the vessel wall, and allowing the inflated cuff to deflate spontaneously over time.

Another alternative method for supporting a surgical component for placement at a surgical site, comprises the steps of gaining access to a surgical site, delivering a surgical component having an inflatable cuff held in direct but loose association by at least one attachment element, or in an alternative embodiment, a plurality of finger elements that may be rigid, to the surgical site, inflating the cuff through a lumen extending from a point of distal egress to the cuff, thereby expanding the surgical component into contact with an intimal wall of a vessel, attaching the surgical component to the vessel wall, and thereafter removing the cuff from the surgical site.

In accordance with an alternative embodiment of the present invention is a method for supporting a surgical component for placement at a surgical site, comprising the steps of gaining access to a surgical site, delivering a surgical component having a distal neck to the surgical site, positioning fin elements of a surgical support apparatus, in conformance with an external surface of a tubular element of the apparatus, delivering the apparatus to a position adjacent to the distal neck of the surgical component, rotating the apparatus to outwardly splay the fin elements, thereby causing the fin elements to contact an inner lumen of the surgical component, further rotating the apparatus to a point at which an outer surface of the surgical component is forced into contact with an intimal wall of a vessel, attaching the surgical component to the vessel wall; rotating the apparatus thereafter to cause the fin elements to conform with the external surface of the tubular element, and removing the apparatus from the surgical site. The step of delivering the apparatus to a position adjacent to the distal neck of the surgical component may comprise the step of delivering the apparatus within a catheter or over a attachment element.

Yet another method for supporting a surgical component for placement at a surgical site, comprises the steps of gaining access to a surgical site, delivering a surgical component having a distal neck to the surgical site, positioning a ring assembly of a surgical support apparatus, which has two or more rings connected by a plurality of helical strands, in conformance with an external surface of a tubular element to which it is attached, delivering the apparatus to a position adjacent to the distal neck of the surgical component, drawing the two or more rings of the ring assembly together, thereby expanding the plurality of helical strands into contact with an inner lumen of the surgical component, further drawing the two or more rings together to force an outer surface of the surgical component into contact with an intimal wall of a vessel, attaching the surgical component to the vessel wall, returning the two or more rings thereafter to a spaced relationship with one another causing the helical strands to re-conform with the external surface of the tubular element, and removing the apparatus from the surgical site. The step of delivering the apparatus to a position adjacent to the proximal neck of the surgical component may comprise the step of delivering the apparatus within a catheter or over a attachment element.

An alternative embodiment of the present invention is directed to a method for supporting a surgical component for placement at a surgical site, comprising the steps of gaining access to a surgical site, delivering the surgical component having a distal neck to the surgical site, holding a cuff of a surgical support apparatus, which has a plurality of spokes, in conformance with an external surface of a tubular element by a conical tip that surrounds the cuff, delivering the apparatus to a position adjacent to the distal neck of the surgical component, advancing the conical tip distally, thereby facilitating the deployment of the plurality of spokes of the cuff, drawing the conical tip proximally causing circumferential expansion of the cuff, thereby forcing the spokes into contact with an inner lumen of the surgical component and the surgical component into contact with an intimal wall of a vessel, attaching the surgical component to the vessel wall, returning the spokes of the cuff thereafter into conformance with the external surface of the tubular element with the conical tip encapsulating the cuff, and removing the apparatus from the surgical site.

It is to be understood that both the foregoing general description and the following detailed description are exemplary and explanatory only, and are not restrictive of the invention as claimed. The accompanying drawings, which are incorporated herein by reference, and which constitute a part of this specification, illustrate certain embodiments of the invention and, together with the detailed description, serve to explain the principles of embodiments of the present invention.

### BRIEF DESCRIPTION OF THE DRAWINGS

In order to assist the understanding of this invention, reference will now be made to the appended drawings, in which like reference characters refer to like elements. The drawings are exemplary only, and should not be construed as limiting the invention.

FIG. 1A is a cross-sectional view of an embodiment of the apparatus of the present invention comprising an expandable cuff at the distal neck of a surgical component.

FIG. 1B is a cross-sectional view of a vessel containing an embodiment of the apparatus of the present invention in an infrarenal positioning (below renal arteries).

FIG. 1C is a cross-sectional view of a vessel containing an embodiment of the apparatus of the present invention having a surgical component and an associated expandable cuff positioned suprarenally (above renal arteries).

FIG. 1D is an illustration of the ring assembly of a further embodiment of the apparatus of the present invention.

FIG. 1E is an illustration of the deployed ring assembly of embodiment at FIG. 1D.

FIG. 2A is a perspective view of an embodiment of the apparatus of the present invention having fin elements conformed to a tubular introducer element.

FIG. 2B is an illustration of the outward splaying of the fin elements upon rotation of the apparatus according to an embodiment of the present invention.

FIG. 2C is a cross-sectional view of a vessel containing an embodiment of the apparatus having fin elements conformed to a tubular element.

FIG. 2D is a cross-sectional view of a vessel containing an embodiment of the apparatus of the present invention with outwardly splayed fin elements in an infrarenal positioning.

FIG. 3A is a perspective view of an embodiment of the apparatus of the present invention with a conical tip encapsulating a cuff.

FIG. 3B is an illustration of the distal advancement of a conical tip of the apparatus according to an embodiment of the present invention.

FIG. 3C is an illustration of the proximal adjustment of the conical tip of the apparatus according to an embodiment of the present invention.

FIG. 3D is a cross-sectional view of a vessel containing the apparatus with the plurality of spokes deployed according to an embodiment of the present invention in an infrarenal positioning.

### DETAILED DESCRIPTION OF EMBODIMENTS OF THE INVENTION

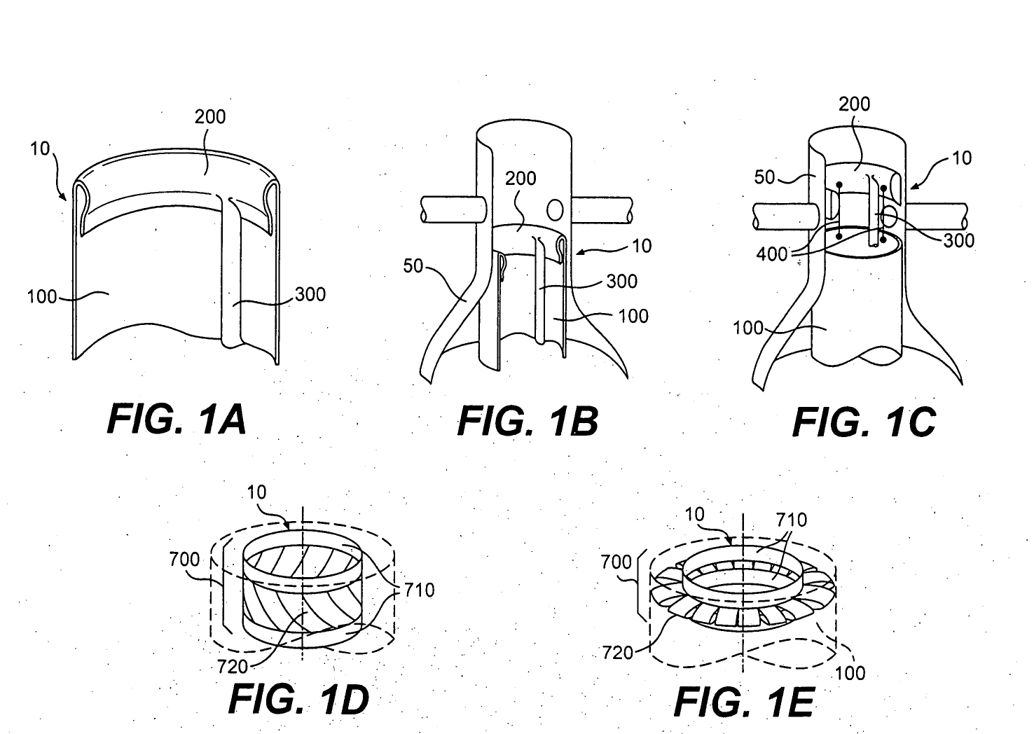

Reference will now be made in detail to embodiments of the present invention, examples of which are illustrated in the accompanying drawings. With reference to FIGS. 1A and 1B, an embodiment of the apparatus 10 for supporting a surgical component comprises a surgical component 100, such as, but not limited to, a prosthetic graft, an expandable cuff 200 located at the distal neck of the component 100, and an integrated lumen 300 within the composition of the component 100. The lumen 300 leads to the cuff 200 to provide for inflation of the cuff 200.

The cuff 200 may be inflated by any suitable means, such as, but not limited to, a compressed gas, compressed gas mixture, or a fluid. After a surgical procedure, the cuff 200 may be deflated or the contents of the inflated cuff may be permitted to leach out over time.

In an alternative embodiment of the present invention, as shown in FIG. 1C, the apparatus 10 for supporting a surgical component comprises an expandable cuff 200 incorporating a lumen 300 which is held in direct but loose association with a surgical component 100, such as, but not limited to, a prosthetic graft, by at least one attachment element, or in an alternative embodiment, a plurality of finger elements 400 that may be rigid. The integrated lumen 300 extends from a point of distal egress to the cuff 200, allowing an interventionist to inflate the cuff 200. The cuff 200 may be inflated by any suitable means, such as a compressed gas, compressed gas mixture, or a fluid or any other suitable inflation means. In addition, the expandable cuff 200 may be located at a suprarenal position.

An embodiment of the apparatus 10 may also include detailing at the central axis of the cuff 200. Certain related surgical components are either supported by or attached to the detailing.

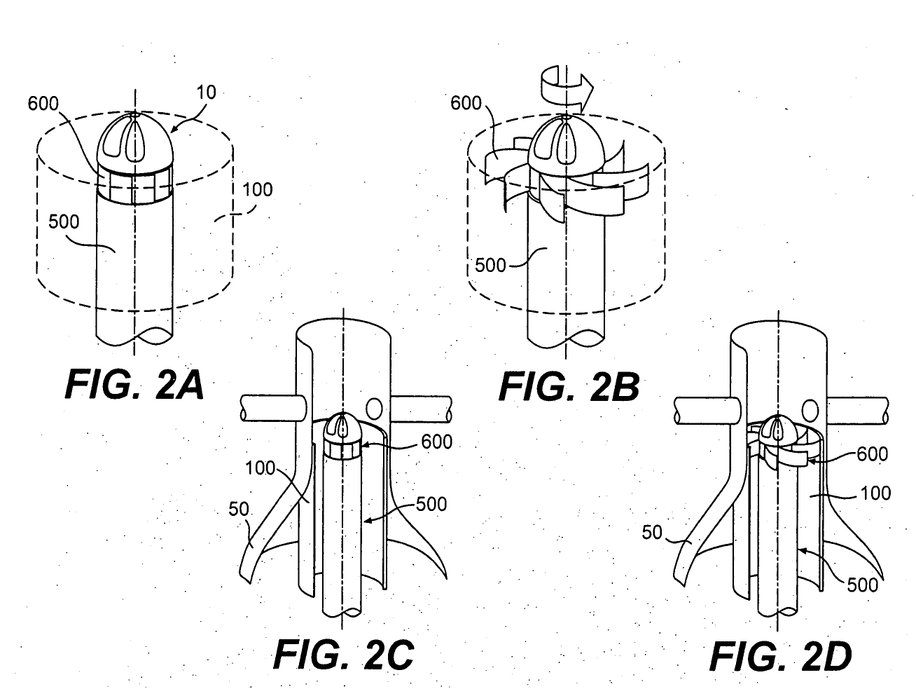

FIGS. 2A through 2D depict an alternative embodiment of the present invention. The apparatus 10 for supporting a surgical component comprises a tubular element 500 and a plurality of fin elements 600 attached about the external surface of the tubular element 500 having conical tip detailing 820. The plurality of fin elements 600 may be attached at uniform spacing about the tubular element 500.

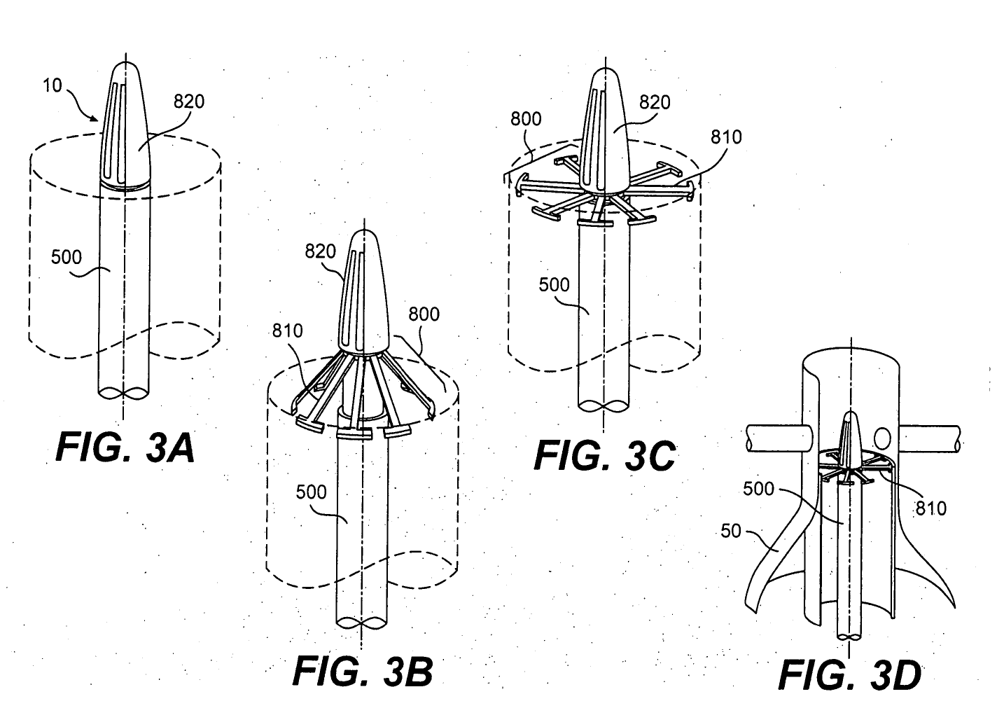

The fin elements 600 conform to the external surface of the tubular element 500 when wound about it for the purpose of insertion into the vessel. Rotation or other manipulation of the tubular element 500 and or conical tip 820 radially deploys the fin elements 600 to support a surgical component 100, such as, but not limited to, a prosthetic graft, within a vessel. Reversing the direction of rotation or manipulation of tubular element 500 forces the fin elements 600 to conform once again to the external surface of the tubular element 500, to enable removal of the apparatus 10 from the surgical site or its repositioning. In a derivative design, the conical tip 820 of tubular component 500 may be adapted to temporarily encapsulate fin elements 600 until they are deployed to aid positioning of surgical component 100 within a vessel as design illustrated in FIGS. 3A-3D.

In another embodiment of the present invention, as depicted in FIGS. 1D and 1E, the apparatus 10 for supporting a surgical component 100 comprises a ring assembly 700, which is adjustably attached to a tubular element (omitted for clarity). The ring assembly 700 includes two or more ring elements 710 that are spaced at a distance from one another and are connected by a plurality of helical strands 720. The helical strands 720 may be, but are not limited to, wires, bands, coils, ribbons, or any other suitable structure.

The positioning of the ring assembly 700 may be adjusted from one in which the rings 710 are at a distance from one another with the strands 720 conforming to the tubular element, to one in which the helical strands 720 splay outwards when rings 710 are drawn together so firmly positioning the surgical component 100 within the subject vessel. The ring assembly 700 may be comprised of any biocompatible metals, such as nitinol, stainless steel, or any other suitable materials. Alternatively, the ring assembly 700 may be comprised of plastic material, such as nylon, delrin, or any other biocompatible materials.

In another alternative embodiment of the present invention, as shown in FIGS. 3A through 3D, the apparatus 10 for supporting a surgical component includes a tubular element 500, which may have a stepped diameter. The apparatus also includes a positioning cuff 800, comprising a plurality of spokes 810 which is slidably located about tubular element 500. The spokes 810 may radiate at a uniform spacing about the tubular element 500. The apparatus 10 further includes a conical tip 820 axially aligned with the tubular element 500. The conical tip 820 selectively encapsulates the cuff detail 800 as shown in FIG. 3A.

The positioning of the conical tip 820 may be adjusted by an interventionist using a remote controller, such as a hand-piece. The tip 820 also may be positioned in a distal, advanced location for facilitating deployment of the spokes 810 of the cuff 800, as depicted in FIG. 3B. Further, the tip 820 may be positioned so that the spokes 810 are firmly held in a circumferentially expanded positioning as shown in FIG. 3C.

The positioning cuff 800 may comprise plastic or metal or any other suitable biocompatible material having an appropriate spring coefficient. The cuff 800 may comprise a single component of unitary construction. Alternatively, the cuff 800 may comprise an assembly of individual components.

A method according to an embodiment of the present invention may comprise the steps of gaining access to a surgical site, delivering a support apparatus 10 to the surgical site, delivering a surgical component 100 to the surgical site, wherein the support apparatus 10 may be incorporated into, or otherwise attached to, or completely separate, from the surgical component 100, expanding an expandable cuff of the support apparatus into contact with a surface, and supporting the surgical component at the surgical site during a surgical procedure. The operation of an embodiment of the apparatus 10 for supporting a surgical component will now be described in connection with FIGS. 1A and 1B. Access to a surgical site is gained. Access may be gained by a catheter, as disclosed in Tanner, et al., U.S. Pat. No. 5,972,023, for Implantation Device for an Aortic Graft Method of Treating Aortic Aneurysm, or a delivery catheter assembly, as disclosed in U.S. patent application Ser. No. 09/783,313, filed Feb. 15, 2001, which are incorporated herein by reference in their entirety. A surgical component 100 having an expandable cuff 200 is delivered to and positioned at the site. The expandable cuff 200 may be located at a distal neck of the surgical component 100. The surgical component may be delivered to the site by a number of various methods, such as those disclosed in Trout, U.S. Pat. No. 5,207,695, for Aortic Graft, Implantation Device, and Method for Repairing Aortic Aneurysm; and Tanner '023 (described above), which are incorporated herein in their entirety by reference. Once the surgical component 100 is temporarily positioned at the surgical site, the cuff 200 is inflated through an integral lumen 300 in the surgical component 100. The cuff 200 may be inflated by a compressed gas mixture, a fluid or any other suitable inflation means. The inflation expands the surgical component 100 into contact with the tissue 50, which may be the intimal wall of a vessel.

The surgical component 100 is then attached to the tissue 50, which may be a vessel wall. The attachment may occur through a number of methods, such as, but not limited to: implanting attachment means having base means, post means, and hook means in a vessel through the use of a balloon catheter system, as disclosed in Trout '695; deploying surgical staples into a component and vessel, as disclosed in Tanner '023; forming a treatment specific hole and inserting a fastener assembly comprising a coiled spring or ring-type fastener through the hole, as disclosed in Tanner et al., U.S. Pat. No. 5,957,940, for Fasteners for Use in the Surgical Repair of Aneurysms; or securing the surgical component and tissue together by an electrically activated thermally inserted fastener assembly, as disclosed in Tanner et al., U.S. Pat. No. 6,248,118, for Heat Activated Surgical Fastener, which are all incorporated herein by reference or any other means to attach the component 100 to a tissue. Following attachment, the cuff 200 may be deflated, or alternatively, its contents may be allowed to leach out over time.

In accordance with the illustration in FIG. 1C, the operation of an alternative embodiment of the apparatus 10 will now be described. Access to a surgical site is gained. A surgical component 100 that has an expandable cuff 200 held in direct but loose association with the component by at least one attachment element, or in an alternative embodiment, a plurality of finger elements 400 that may be rigid is delivered to the surgical site. The cuff 200 is inflated through an integratal lumen 300, thereby expanding the cuff 200 and the surgical component 100 into contact with the tissue, which may be the intimal wall of a vessel. The cuff 200 may be inflated by a compressed gas, compressed gas mixture, a fluid or any other suitable inflation means. The surgical component 100 is then attached to the tissue, which may be a vessel wall. Once the attachment is complete, the cuff 200 is withdrawn from the component 100 and thereafter removed from the surgical site.

The operation of an alternative embodiment of the apparatus 10, as shown in FIGS. 2A through 2D will now be described. Access to a surgical site is gained. A surgical component 100 is delivered to and positioned at the surgical site. Fin elements 600 of the apparatus 10 are held in conformance with the external surface of a central tubular element 500 of the apparatus 10, as depicted in FIG. 2A. The apparatus 10 is then delivered to the surgical site to a position adjacent to the distal neck of the surgical component 100. The apparatus 10 may be delivered within a catheter or over a attachment element or by any other suitable means. The apparatus 10 is rotated, or manipulated in any other suitable way, which splays the fin elements 600, as illustrated in FIG. 2B. The rotation or manipulation causes the fin elements 600 to contact the inner lumen of the surgical component 100, as shown in FIG. 2D. The apparatus 10 is further rotated or manipulated forcing an outer surface of the surgical component 100 into contact with the intimal wall of a vessel. The surgical component 100 may be attached to a vessel wall, by any suitable means. The apparatus 10 is rotated or manipulated to cause the fin elements 600 to return to conformance with the external surface of the tubular element 500. The apparatus 10 is then removed from the surgical site.

In accordance with FIGS. 1D and 1E, the operation of another alternative embodiment of the apparatus 10 of the present invention will now be described. Access to a surgical site is gained and a surgical component 100 is delivered to and positioned at the site. A ring assembly 700 of the apparatus 10 is positioned in conformance with the external surface of a central tubular element to which it is attached. In this position, two or more rings 710 of the ring assembly 700 are spaced at a distance from one another, connected by a plurality of helical strands 720, as shown in FIG. 1D. The apparatus 10 is then delivered to the surgical site at a position adjacent to the distal neck of the surgical component. The apparatus 10 may be delivered within a catheter or over a attachment element or by any other suitable means. The two or more rings 710 of the ring assembly 700 are drawn toward one another, thereby expanding the helical strands 720 that connect the rings 710 into contact with the inner lumen of the surgical component, as shown in FIG. 1E. The rings 710 may be drawn together by a hand controller that is connected to the apparatus 10 or by any other suitable means. The hand controller may be operated by an interventionist. The rings 710 are then further drawn together, forcing the outer surface of the surgical component into contact with the tissue, which may be the intimal wall of a vessel. The surgical component is then attached to the tissue, by any suitable means. Once attachment is complete, the two or more rings 710 of the ring assembly 700 are returned to a spaced relationship, causing the helical strands 720 to conform to the external surface of the tubular element. The apparatus 10 is removed from the surgical site.

The operation of another alternative embodiment of the apparatus 10, as shown in FIGS. 3A through 3D, will now be described. Access to a surgical site is gained and a surgical component is delivered to and positioned at the site. A positioning cuff 800, which has a plurality of spokes 810, of the apparatus 10 is held in conformance with the external surface of a central tubular element 500 by a hollow conical tip 820. Once positioned, the apparatus 10 is delivered to the surgical site at a position adjacent to the distal neck of the surgical component. The conical tip 820 of the apparatus 10 is advanced distally, deploying the radial spokes 810 of the cuff 800, as depicted in FIG. 3B. The tip 820 is then drawn proximally, which causes further expansion of the cuff 800 as shown in FIG. 3C. This expansion forces the spokes 810 into contact with the inner lumen of the surgical component and the surgical component into contact with the tissue, which may be the intimal wall of a vessel. The movement of the tip 820 may be controlled by a hand controller that is connected to the apparatus 10 and operated by an interventionist or by any other suitable means. Once the surgical component 100 is forced into contact with the tissue, it is attached thereto by any suitable means. The spokes 810 of the cuff 800 are then returned to conformance with the tubular element 500, the conical tip 820 encapsulating the cuff 800. The apparatus 10 is then removed from the surgical site.

It will be apparent to those skilled in the art that variations and modifications of embodiments of the present invention can be made without departing from the scope or spirit of the invention. For example, the method for supporting a surgical component for placement at a surgical site could be used in settings other than the repair of aneurysms. The method could be used in the attachment of any prosthetic material to any tissue with a metal or plastic attachment device such as shape memory metal or a plastic or metal staple. For instance, the method could be used during the attachment of a prosthetic mesh to fascia through a laparoscope/endoscope or directly in an open operation for hernia repair. Thus, it is intended that embodiments of the present invention cover all such modifications and variations of the invention, provided they come within the scope of the appended claims and their equivalents.

What is claimed is:

1. An apparatus for supporting a surgical component, comprising:
an expandable cuff; and
expansion means for expanding the cuff to contact a surface to support a surgical component.

2. The apparatus according to claim 1, wherein the expandable cuff is incorporated into the surgical component.

3. The apparatus according to claim 1, wherein the expandable cuff is attached to the surgical component by at least one attachment element.

4. The apparatus according to claim 3, wherein the expandable cuff is removably attached to the surgical component.

5. The apparatus according to claim 1, wherein the expandable cuff is inflatable.

6. The apparatus according to claim 1, further comprising a lumen attached to the expandable cuff.

7. The apparatus according to claim 1, wherein the expandable cuff comprises a ring assembly.

8. The apparatus according to claim 7, wherein the ring assembly further comprises at least two ring elements and at least two helical strands.

9. The apparatus according to claim 1, wherein the expandable cuff comprises a tubular element and fins.

10. The apparatus according to claim 1, wherein the expandable cuff comprises spokes and a conical tip.

11. An apparatus for supporting a surgical component during a surgical procedure, comprising:
an expandable cuff, wherein the expandable cuff is inflatable;
a lumen with a first end and a second end, wherein the first end is attached to the expandable cuff; and
expansion means attached to the second end of the lumen for expanding the expandable cuff into contact with a surface to support a surgical component.

12. The apparatus according to claim 11, wherein the expandable cuff is incorporated into the surgical component.

13. The apparatus according to claim 11, wherein the expandable cuff is attached to the surgical component by at least one attachment element.

14. An apparatus for supporting a surgical component during a surgical procedure, comprising:
an expandable cuff comprising a ring assembly; and
expansion means for expanding the cuff to contact a surface to support a surgical component.

15. The apparatus according to claim 14, wherein the ring assembly further comprises:
a first and a second ring element; and
at least two helical strands with a first end and a second end,
wherein the first end is attached to the first ring element and the second end is attached to the second ring element.

16. An apparatus for supporting a surgical component during a surgical procedure, comprising:
an expandable cuff comprising a tubular element and at least one fin, wherein the at least one fin has a first end and a second end, wherein the first end is substantially free and passes through an opening in the tubular element; and
expansion means attached to the second end of the at least one fin for expanding the first end of the at least one fin into contact with a surface to support a surgical component.

17. An apparatus for supporting a surgical component during a surgical procedure, comprising:
an expandable cuff comprising a conical tip and a spoke assembly with a first and a second configuration, wherein the spoke assembly is disposed within the conical tip in the first configuration; and
wherein the spoke assembly further comprises at least one spoke with a first end and a second end, wherein the first end is substantially free; and
expansion means for expanding the spoke assembly into a second configuration wherein the first end of the at least one spoke contacts a surface to support a surgical component.

18. The apparatus according to claim 17, wherein the second end of the at least one spoke is attached to the expansion means.

19. The apparatus according to claim 17, wherein the conical tip is attached to the expansion means.

20. A method for supporting a surgical component for placement at a surgical site, comprising the steps of:
gaining access to a surgical site;
delivering a support apparatus to the surgical site;
delivering a surgical component to the surgical site;
expanding an expandable cuff of the support apparatus into contact with a surface;
supporting the surgical component at the surgical site during a surgical procedure.

21. The method according to claim 20, wherein the support apparatus and surgical component are delivered to the surgical site in the same step.

22. The method according to claim 20, wherein the step of expanding the expandable cuff further comprises the step of inflating the expandable cuff.

23. The method according to claim 20, wherein the step of expanding the expandable cuff further comprises the step of drawing at least two ring elements together to expand at least two helical strands into contact with a surface.

24. The method according to claim 20, wherein the step of expanding the expandable cuff further comprises the step of expanding at least one fin into contact with a surface.

25. The method according to claim 20, further comprising the step of contracting the expandable cuff to its pre-expansion configuration.

26. The method according to claim 20, further comprising the step of removing the support apparatus from the surgical site following the surgical procedure.

27. The method according to claim 20, further comprising the step of attaching the surgical component to a tissue.

28. The method according to claim 20, further comprising the step of attaching the surgical component to another surgical component.
